(19) United States
(12) Patent Application Publication
(10) Pub. No.: US 2026/0215803 A1
(43) Pub. Date: Jul. 30, 2026

(54) RING PUNCH FOR HARVESTING MUCOSAL GRAFTS

(71) Applicant: Helmut Zepf Medizintechnik GmbH, Seitingen-Oberflacht (DE)

(72) Inventor: Patrick ZEPF, Seitingen-Oberflacht (DE)

(21) Appl. No.: 19/573,625

(22) Filed: Mar. 20, 2026

(30) Foreign Application Priority Data
Mar. 24, 2020 (DE) 202025101571.4

(51) Int. Cl. A61B 17/3205

(52) U.S. Cl. CPC A61B 17/32053

(57) ABSTRACT

A ring punch for harvesting mucosal grafts with an outer cylindrical sleeve having an outer wall, a first outer end, a second outer end, a first longitudinal axis and a first inner diameter and an inner cylindrical sleeve with an inner wall, a first inner end, a second inner end, a second longitudinal axis and a second inner diameter. The inner cylindrical sleeve is arranged concentrically in the outer cylindrical sleeve. A first cutting edge is arranged on the first outer end of the outer cylindrical sleeve and a second cutting edge is arranged on the first inner end of the inner cylindrical sleeve. A radially protruding projection is spaced from the first outer end of the outer cylindrical sleeve by a distance of two to four millimeters at least in sections on an outer surface of the outer wall.

The invention relates to a ring punch for harvesting mucosal grafts according to the preamble of claim 1.

Extracting a tooth and replacing the missing tooth with a dental implant are typically followed by a remodeling of the surrounding tissue, not only at the bone level, but also in the soft tissue. In many cases, tissue augmentation is also required.

To enable the harvesting of a suitable tissue graft, particularly a mucosal graft, from another location in the mouth, ring punches for harvesting mucosal grafts with an outer cylindrical sleeve with a wall, a first end, a second end, a first longitudinal axis, and a first inner diameter, as well as with an inner cylindrical sleeve with a wall, a first end, a second end, a second longitudinal axis, and a second inner diameter are known, wherein the inner cylindrical sleeve is arranged concentrically in the outer cylindrical sleeve, and wherein a first cutting edge is arranged at the first end of the outer cylindrical sleeve and a second cutting edge is arranged at the first end of the inner cylindrical sleeve. With such a ring punch, a ring-shaped mucosal graft can be harvested and then placed over the implanted tooth.

The problem of the invention is to further develop such a ring punch so that it can be used more safely.

The problem is solved according to the invention by a ring punch with the features of claim 1.

Advantageous embodiments and further developments of the invention are specified in the dependent claims.

In one ring punch according to the invention for harvesting mucosal grafts with an outer cylindrical sleeve with a wall, a first end, a second end, a first longitudinal axis, and a first inner diameter, as well as with an inner cylindrical sleeve with a wall, a first end, a second end, a second longitudinal axis, and a second inner diameter, wherein the inner cylindrical sleeve is arranged concentrically in the outer cylindrical sleeve, and wherein a first cutting edge is arranged on the first end of the outer cylindrical sleeve and a second cutting edge is arranged on the first end of the inner cylindrical sleeve, a radially protruding projection is arranged at least in sections on an outer surface of the wall of the outer cylindrical sleeve, wherein this projection is spaced from the first end of the outer cylindrical sleeve by a distance of at least 2 mm and at most 4 mm. Such a radially protruding projection forms a depth stop that can reliably prevent tissue harvesting that is too deep. This enables, in particular, the safe harvesting of mucosal grafts even in the palate area without the risk of damaging blood vessels.

In an especially preferred way, the distance between the projection and the first end is approximately 3 mm, whereby tissue harvesting that is too deep can be reliably prevented for typical anatomical configurations.

According to one especially preferred further development of the invention, the projection has a circumferential structure, further increasing safety.

If the projection has a circumferential structure, it is thus shaped like a ring. It is possible to slip the projection over the outer cylindrical sleeve or to place it against the outside of this sleeve and to secure the projection by gluing, pressing, or lasering.

Preferably, however, the projection is integrally formed on the outer cylindrical sleeve. This enables more precise relative alignment between the projection and the first end of the outer cylindrical sleeve.

According to one alternative preferred embodiment, the projection is adjustably arranged on the outer cylindrical sleeve, in particular, it can be displaced in the direction parallel to the first longitudinal axis of the outer cylindrical sleeve. This enables adaptation to anatomical conditions. The adjustability can be achieved, for example, by a threaded connection, a snap-fit connection, or a clamping connection between the projection and the outer cylindrical sleeve.

Advantageously, the projection has a dark color. This makes the projection more clearly visible when working in the oral cavity.

One especially advantageous embodiment provides that at least one opening, preferably two diametrically opposed openings, are arranged in the wall of the outer cylindrical sleeve in the area between the projection and the second end of the outer cylindrical sleeve. The opening thus leads into the space between the outer cylindrical sleeve and the inner cylindrical sleeve, but due to its arrangement between the projection and the second end of the outer cylindrical sleeve, it is positioned behind a mucosal graft harvested with the ring punch when viewed from the first end of the cylindrical sleeve, so that a tool can be inserted through the opening into the space and, by applying pressure to the mucosal graft, eject it from the ring punch.

Because the ring punch is used in the oral cavity, the ring punch is advantageously made of medical-grade steel, which is both biocompatible and easy to clean, in particular, suitable for sterilization.

Preferably, the second end of the outer cylindrical sleeve and the second end of the inner cylindrical sleeve are arranged on a cover part, thereby enabling the two sleeves to be fixed relative to each other in a structurally simple way. On the side of the cover part facing away from the sleeves, a drive rod is arranged, which preferably has a non-circular section on one free end. The non-circular section can be connected to a hand grip or a motor-driven drive, to be able to rotate the ring punch around the longitudinal axis either manually or by motor, thereby making it easier to cut the mucosal graft out of the tissue.

Preferably, the height of the radial projection is approximately 2 mm and/or the axial length of the radial projection is approximately 1 mm. Such dimensions have proven to be particularly suitable for the use of the ring punch in the oral cavity.

Preferably, the inner diameter of the inner cylindrical sleeve is 3 mm to 5 mm, preferably approximately 4 mm. The inner diameter of the inner cylindrical sleeve determines the size of the hole in the mucosal graft, through which the dental implant, around which the tissue is to be built up, protrudes. Such a size is well-suited for typical applications.

Preferably, the inner diameter of the outer cylindrical sleeve is 6 mm to 12 mm, for example, approximately 7 mm, approximately 9 mm, or approximately 11 mm. Depending on the area around the dental implant where tissue is to be built up, different sizes can be used.

Preferably, the length of the outer cylindrical sleeve is approximately 15 mm to 20 mm to enable good handling in the oral cavity.

One embodiment of the invention will be explained in detail with reference to the following figures. Shown are

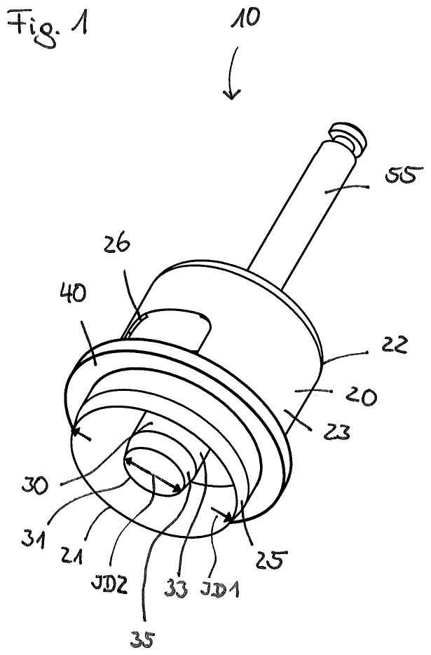

FIG. 1 a perspective view of an embodiment of a ring punch according to the invention,

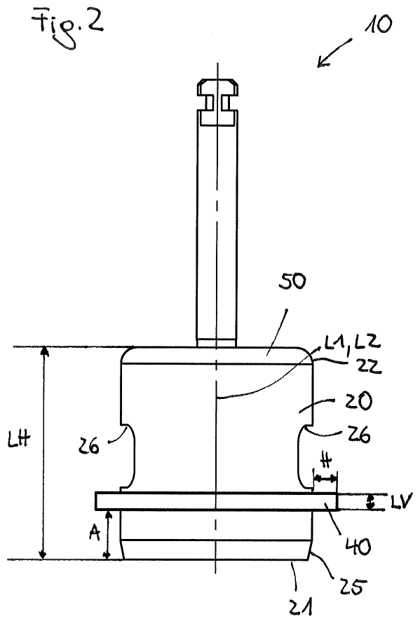

FIG. 2 a side view of the ring punch according to FIG. 1, and

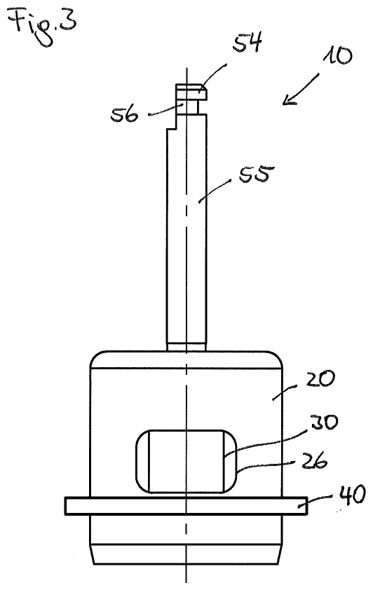

FIG. 3 another side view of the ring punch according to FIG. 1.

FIGS. 1 to 3 show different views of one embodiment of a ring punch 10 according to the invention for harvesting mucosal grafts. For better visibility, not all reference symbols are shown in all figures.

The ring punch 10 has an outer cylindrical sleeve 20 with a wall 23, a first end 21, a second end 22, a first longitudinal axis L1, and a first inner diameter ID1, as well as an inner cylindrical sleeve 30 with a wall 33, a first end 31, a second end, a second longitudinal axis L2, and a second inner diameter ID2, wherein the inner cylindrical sleeve 30 is arranged concentrically in the outer cylindrical sleeve 20. In this way, in particular, the first longitudinal axis L1 and the second longitudinal axis L2 coincide.

The inner diameter ID2 of the inner cylindrical sleeve 30 can be in the range from 3 mm to 5 mm and preferably approximately 4 mm. The inner diameter ID1 of the outer cylindrical sleeve 20 can be in the range from 6 mm to 12 mm and, for example, approximately 7 mm, approximately 9 mm, or approximately 11 mm.

A length LH of the outer cylindrical sleeve can be approximately 15 mm to 20 mm.

The second end 22 of the outer cylindrical sleeve 20 and the second end of the inner cylindrical sleeve 30 can be arranged on a cover part 50. A drive rod 55, which has a non-circular section 55 preferably on a free end 54, can be arranged on the side of the cover part 50 facing away from the sleeves 20, 30.

In particular, the two sleeves 20, 30 are arranged relative to each other such that the first end 21 of the outer cylindrical sleeve 20 and the second end 31 of the inner cylindrical sleeve 30 lie in one plane.

A first cutting edge 25 is arranged on the first end 21 of the outer cylindrical sleeve 20 and a second cutting edge 35 is arranged on the first end 31 of the inner cylindrical sleeve 30. The cutting edges 25, 35 can be formed, for example, by a chamfer. In particular, the chamfer is arranged on an outer surface of the wall 23, 33 of the respective sleeve 20, 30.

The cutting edge 25, 35 can extend over an axial length of approximately 1 to 2 mm.

A radially protruding projection 40, which is spaced from the first end 21 of the outer cylindrical sleeve 20 by a distance A of at least 2 mm and at most 4 mm, preferably approximately 3 mm, is arranged on at least in sections on an outer surface of the wall 23 of the outer cylindrical sleeve 20. This projection 40 forms a depth stop and enables the ring punch 10 to penetrate into the tissue by a maximum distance A.

In the present embodiment, the projection 40 has a circumferential structure and is thus shaped like a ring. The projection 40 can be manufactured as a separate part and then be attached to the outer surface of the wall 23. Preferably, however, the projection 40 is arranged integrally on the outer cylindrical sleeve 20. The projection 40 has a height H that can be approximately 2 mm. The projection can extend over an axial length LV of approximately 1 mm. Furthermore, the projection 40 can have a dark coloring to make it more easily visible in the oral cavity.

At least one opening 26, preferably two diametrically opposed openings 26, can be arranged in the wall 23 of the outer cylindrical sleeve 20 in the area between the projection 40 and the second end 22. Here, it is advantageous if the opening 26, in particular, the two openings 26, are arranged as close as possible to the projection 40. A tool can be inserted through these openings 26 into the space between the outer cylindrical sleeve 20 and the inner cylindrical sleeve 30 and thereby pushing a mucosal graft located in this space out of the ring punch 10 toward the first end 21 of the outer cylindrical sleeve 20.

The ring punch 10 is made from medical-grade steel, for example, from martensitic stainless steel.

### List of Reference Symbols

10 Ring punch
20 Outer cylindrical sleeve
21 First end
22 Second end
23 Wall
25 First cutting edge
26 Opening
30 Inner cylindrical sleeve
31 First end
33 Wall
35 Second cutting edge
40 Projection
40 Cover part
54 Free end
55 Drive rod
56 Non-circular section
ID1 First inner diameter
ID2 Second inner diameter
L1 First longitudinal axis
L2 Second longitudinal axis
A Distance
H Height
LV Length
LH Length

1. A ring punch for harvesting mucosal grafts, the ring punch comprising:
an outer cylindrical sleeve with an outer wall, a first outer end, a second outer end, a first longitudinal axis and a first inner diameter;
an inner cylindrical sleeve with an inner wall, a first inner end, a inner second end, a second longitudinal axis and a second inner diameter, wherein the inner cylindrical sleeve is arranged concentrically in the outer cylindrical sleeve, and wherein a first cutting edge is arranged on the first outer end of the outer cylindrical sleeve and a second cutting edge is arranged on the first inner end of the inner cylindrical sleeve; and
a radially protruding projection that is spaced from the first outer end of the outer cylindrical sleeve by a distance of at least two millimeters (2 mm) and at most four millimeters (4 mm), the radially protruding projection is arranged at least in sections on an outer surface of the outer wall of the outer cylindrical sleeve.

2. The ring punch according to claim 1, wherein the distance is approximately three millimeters (3 mm).

3. The ring punch according to claim 1, characterized in that the radially protruding projection has a circumferential structure.

4. The ring punch according to claim 1, characterized in that the radially protruding projection is arranged integrally on the outer cylindrical sleeve.

5. The ring punch according to claim 1, characterized in that the radially protruding projection is arranged so that the radially protruding projection can be adjusted on the outer cylindrical sleeve.

6. The ring punch according to claim 1, characterized in that the projection has a dark color.

7. The ring punch according to claim 1, characterized in that an opening (26) is arranged in the outer wall in an area between the projection and the second outer end.

8. The ring punch according to claim 1 characterized in that the ring punch is constructed of medical-grade steel.

9. The ring punch according to claim 1 characterized in that the second outer end and the second inner end are arranged on a cover part, wherein a drive rod that has a non-circular section on a free end is arranged on a side of the cover part facing away from the outer and inner cylindrical sleeves.

10. The ring punch according to claim 1, characterized in that a height of the radially protruding projection is approximately two millimeters (2 mm).

11. The ring punch according to claim 1 characterized in that the second inner diameter is three to five millimeters (3 mm to 5 mm).

12. The ring punch according to claim 1, characterized in that the first inner diameter is six to twelve millimeters (6 mm to 12 mm).

13. The ring punch according to claim 1, characterized in that an length of the outer cylindrical sleeve is approximately fifteen to twenty millimeters (15 mm to 20 mm).

14. The ring punch according to claim 7, wherein the opening is comprised of two diametrically opposed openings.

15. The ring punch according to claim 1, wherein an axial length of the radially protruding projection is approximately one millimeter (1 mm).

16. The ring punch according to claim 11, wherein the second inner diameter is approximately four millimeters (4 mm).

17. The ring punch according to claim 12, wherein the first inner diameter is approximately seven millimeters (7 mm).

18. The ring punch according to claim 12, wherein the first inner diameter si approximately nine millimeters (9 mm).

19. The ring punch according to claim 12, wherein the first inner diameter is approximately eleven millimeters (11 mm).
